(19) United States
(12) Patent Application Publication
(10) Pub. No.: US 2024/0164721 A1
(43) Pub. Date: May 23, 2024

(54) METHOD AND APPARATUS FOR DETERMINING MEAL START AND PEAK EVENTS IN ANALYTE MONITORING SYSTEMS

(71) Applicant: ABBOTT DIABETES CARE INC., Alameda, CA (US)

(72) Inventors: Erwin Satrya Budiman, Fremont, CA (US); Gary Alan Hayter, Oakland, CA (US)

(21) Appl. No.: 18/212,075

(22) Filed: Jun. 20, 2023

Related U.S. Application Data
(63) Continuation of application No. 16/703,196, filed on Dec. 4, 2019, now Pat. No. 11,717,225.
Application No. 16/703,196 is a continuation of application No. 15/300,711, filed on Sep. 29, 2016, now abandoned.
(60) Provisional application No. 61/972,381, filed on Mar. 30, 2014.

(51) Int. Cl. A61B 5/00; A61B 5/145; G16H 20/60; G16H 50/20

(52) U.S. Cl. CPC A61B 5/7203; A61B 5/145; A61B 5/14532; A61B 5/7282; G16H 20/60; G16H 50/20

(57) ABSTRACT

Systems, methods and apparatus are provided for estimating meal start and peak meal response times are provided based on time series of sampled glucose data collected. Numerous additional aspects are disclosed.

### CROSS-REFERENCE TO RELATED APPLICATIONS

The present application is a continuation of U.S. patent application Ser. No. 16/703,196, filed Dec. 4, 2019, which is a continuation of U.S. patent application Ser. No. 15/300,711, filed Sep. 29, 2016, now abandoned, which is a national stage patent application under 35 U.S.C. § 371 claims priority to PCT Application Serial No. PCT/US2015/23380, filed Mar. 30, 2015, which claims priority to and the benefit of U.S. Provisional Patent Application Ser. No. 61/972,381, filed Mar. 30, 2014, all of which are incorporated by reference herein in their entireties for all purposes.

### BACKGROUND

The detection of the concentration level of glucose or other analytes in certain individuals may be vitally important to their health. For example, the monitoring of glucose levels is particularly important to individuals with diabetes or pre-diabetes. People with diabetes may need to monitor their glucose levels to determine when medication (e.g., insulin) is needed to reduce their glucose levels or when additional glucose is needed.

Devices have been developed for automated in vivo monitoring of analyte time series characteristics, such as glucose levels, in bodily fluids such as in the blood stream or in interstitial fluid. Some of these analyte level measuring devices are configured so that at least a portion of a sensor of an on-body device is positioned below a skin surface of a user, e.g., in a blood vessel or in the subcutaneous tissue of a user. As used herein, the term analyte monitoring system is used to refer to any type of in vivo monitoring system that uses a sensor disposed with at least a subcutaneous portion to measure and store sensor data representative of analyte concentration levels automatically over time.

Existing approaches to determining pre-prandial and post-prandial meal responses are achieved in several ways. One way to determine the pre-prandial and post-prandial meal responses uses paired fingerstick blood glucose tests, where glucose measurements are taken at the start of the meal and at a certain relative duration since the meal start. In this approach, however, the variability of duration between start and peak of meals results in estimation errors of the meal response. Another approach to determine pre-prandial and post-prandial meal responses uses a collection of dense glucose measurements (e.g. once every 10 minutes) in conjunction with user entered meal markers. However, as most meal markers only indicate the start of the meal, the availability and accuracy of such markers are affected by the patient's schedule and other unforeseeable circumstances. Yet another approach to determining pre-prandial and post-prandial meal responses includes collection of dense glucose measurement and a pre-determined time of day window, where glucose values within a particular time of day window are assumed to represent pre-breakfast, post-breakfast, for example. However, in this approach, the reliability of the estimates will largely depend upon the consistency in the patient's meal timing routine.

### SUMMARY

Accordingly, embodiments of the present disclosure provide systems, methods, and apparatus for estimating or detecting start of meal event and peak meal response based on real time or pseudo-retrospective, or retrospective analysis of data corresponding to monitored analyte levels, which can be used to modify insulin therapy regimen such as adjusting the basal delivery rate for pump users, and/or adjusting bolus dose levels.

Certain embodiments of the present disclosure include performing conditioning on a plurality of data points corresponding to monitored analyte level over a first time period, for each data point, determining a time derivative based on the conditioned plurality of data points, determining optima of acceleration based on the determined time derivatives, removing false carbohydrate intake start and peak carbohydrate intake response pairs having an amplitude below a predetermined level, removing carbohydrate intake start candidate from the most current carbohydrate intake peak response candidate, removing unpaired carbohydrate intake start candidates and signal artifact falsely identified as carbohydrate intake start and carbohydrate intake peak response pair, and refining the identified carbohydrate intake start and peak carbohydrate intake response pairs.

Certain embodiments of the present disclosure include a user interface component and one or more processors operatively coupled to the user interface component, the one or more processors configured to perform conditioning on a plurality of data points corresponding to monitored analyte level over a first time period, for each data point, to determine a time derivative based on the conditioned plurality of data points, to determine optima of acceleration based on the determined time derivatives, to remove false carbohydrate intake start and peak carbohydrate intake response pairs having an amplitude below a predetermined level, to remove carbohydrate intake start candidate from the most current carbohydrate intake peak response candidate, to remove unpaired carbohydrate intake start candidates and signal artifact falsely identified as carbohydrate intake start and carbohydrate intake peak response pair, and to remove the identified carbohydrate intake start and peak carbohydrate intake response pairs.

Numerous other aspects and embodiments are provided. Other features and aspects of the present disclosure will become more fully apparent from the following detailed description, the appended claims, and the accompanying drawings.

### BRIEF DESCRIPTION OF THE FIGURES

The accompanying drawings, which are incorporated herein, form part of the specification. Together with this written description, the drawings further serve to explain the principles of, and to enable a person skilled in the relevant arts, to make and use the present disclosure.

FIG. 1 illustrates a flowchart for meal start and peak detection routine in accordance with certain embodiments of the present disclosure;

FIG. 2 illustrates a flowchart for performing time series sampled analyte data conditioning of the meal start and peak detection routine of FIG. 1 in accordance with certain embodiments of the present disclosure;

FIG. 3 illustrates a flowchart for sampled data analysis to remove questionable data of FIG. 2 in accordance with certain embodiments of the present disclosure;

FIG. 4 illustrates a flowchart for data conditioning and/or data recovery for smooth output of FIG. 2 in accordance with certain embodiments of the present disclosure;

FIG. 5 illustrates sample data analysis to remove questionable data and performing condition and/or data recovery for smooth output in conjunction with the routines above in certain embodiments of the present disclosure;

FIG. 6 illustrates data conditioning and/or data recovery for smooth output in conjunction with the routines above in certain embodiments of the present disclosure;

FIG. 7 illustrates determination of backward and forward slopes for peak and meal start candidates in conjunction with the routines above in certain embodiments of the present disclosure;

FIG. 8 illustrates determination of acceleration and the identification of local acceleration optima in conjunction with the routines above in certain embodiments of the present disclosure;

FIG. 9 illustrates an example of removal of adjacent candidates of the same type in conjunction with the routines above in certain embodiments of the present disclosure;

FIG. 10 illustrates examples of removal of false meal start and peak pairs with a small amplitude in conjunction with the routines above in certain embodiments of the present disclosure;

FIG. 11 illustrates removal of unpaired meal start candidates and surviving spike artifacts falsely identified as a meal start/peak pair in conjunction with the routines above in certain embodiments of the present disclosure;

FIG. 12 illustrates refinement of identified meal start and peak instances in conjunction with the routines above in certain embodiments of the present disclosure; and

FIG. 13 illustrates an example of comparison of estimated meal start determination in conjunction with the routines described herein against manually marked meal start events.

### DETAILED DESCRIPTION

Before the embodiments of the present disclosure are described, it is to be understood that this disclosure is not limited to the particular embodiments described, as such may, of course, vary. It is also to be understood that the terminology used herein is for the purpose of describing particular embodiments only, and is not intended to be limiting, since the scope of the embodiments of the disclosure will be limited only by the appended claims.

The present disclosure provides systems, methods, and apparatus to determine meal start and peak events based on analysis of information associated with monitored analyte concentration level. According to embodiments of the present disclosure, a dataset representative of a patient's monitored analyte concentration level (herein referred to as “sensor data”) over time is received from an on-body device that includes sensor electronics operatively coupled to an analyte sensor that is in fluid contact with interstitial fluid. In some embodiments, the sensor data may represent a collection of data received from the on-body device at several different times during a wear period of the on-body device. In some other embodiments, the sensor data may represent data collected and stored over an entire wear period of the on-body device and only received from the on-body device at the end of the wear period or at the end of the useful life of the on-body device. In other words, the sensor data can be transmitted continuously, on a regular schedule, in multiple batches over time, in batches on demand, or in a single batch.

Embodiments of the present disclosure may be applied to any analyte concentration level determination system that may exhibit or at least be suspected of exhibiting, or that may be susceptible to noise in the sensor data. Embodiments of the disclosure are described primarily with respect to continuous glucose monitoring devices and systems, but the present disclosure may be applied to other analytes and analyte characteristics, as well as data from measurement systems that transmit sensor data from a sensor unit to another unit such as a processing or display unit in response to a request from the other unit. For example, other analytes that may be monitored include, but are not limited to, acetyl choline, amylase, bilirubin, cholesterol, chorionic gonadotropin, creatine kinase (e.g., CK-MB), creatine, DNA, fructosamine, glutamine, growth hormones, hormones, ketones, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In the embodiments that monitor more than one analyte, the analytes may be monitored at the same or different times. The present disclosure also provides numerous additional embodiments.

Embodiments of the present disclosure may include a programmed computer system adapted to receive and store data from an analyte monitoring system. The computer system may include one or more processors for executing instructions or programs that implement the methods described herein. The computer system may include memory and persistent storage devices to store and manipulate the instructions and sensor data received from the analyte monitoring system. The computer system may also include communications facilities (e.g., wireless and/or wired) to enable transfer of the sensor data from the analyte monitoring system to the computer. The computer system may include a display and/or output devices for identifying dropouts in the sensor data to a user. The computer system may include input devices and various other components (e.g., power supply, operating system, clock, etc.) that are typically found in a conventional computer system. In some embodiments, the computer system may be integral to the analyte monitoring system. For example, the computer system may be embodied as a handheld or portable receiver unit within the analyte monitoring system.

Embodiments of the present disclosure perform analysis on analyte (e.g., glucose) data collected from analyte monitoring systems that includes a combination of asynchronous real-time and time spaced (e.g., 5 minutes-, 10 minutes-, 15 minutes-, 20 minutes-, 30 minutes-apart historical glucose data such as in flash glucose monitoring (FGM) systems, to be processed for analyte pattern and titration analysis. Embodiments also include analysis of mixed data collected from any of the following systems: discrete blood glucose monitoring (DGM) systems, continuous glucose monitoring (CGM) systems, in addition to flash glucose monitoring (FGM) systems. In this manner, embodiments of the present disclosure provide improved reliability of glucose pattern and titration analysis by distinguishing between true glucose trends and measurement errors, artifacts, and gaps caused by the measurement timing and/or process.

Prior approaches took pre-defined windows of time (e.g. entire 14 day sensor data collected, or 24 hours) to calculate glucose variability and median glucose. Such an approach analyzed each glucose reading independently, regardless of the relative timing and physiological feasibility of the relative magnitudes of glucose values. On the other hand, embodiments of the present disclosure perform physiological feasibility checks by comparing analyte level readings that are spaced close in time to obtain a more reliable estimate of glucose values.

Embodiments of the present disclosure includes first identifying and removing questionable data, where physiological limits are used to compare each measurement in the context of other nearby measurements, and thereafter, performing data conditioning and recovery including, for example, where surviving sampled data are conditioned, by signal processing, to minimize the amount of noise as much as possible, and the removed sampled data are supplemented by data based on other sampled data in close proximity to it in time. For example, performing data conditioning and recovery procedure includes data analysis routines described in pending U.S. patent application Ser. No. 14/210,312 entitled “Noise rejection Methods and Apparatus for Sparsely Sampled Analyte Sensor Data” filed on Mar. 13, 2014, the disclosure of which is incorporated herein by reference for all purposes.

Embodiments of the present disclosure include estimating the start time and peak instances of meal events based on sampled analyte data such as glucose measurement data, which can be used to improve the reliability of existing start-of-meal markers manually entered by the user.

In certain embodiments, the following assumptions are made when the algorithm performs data processing to determine the start and the peak of a meal event. For example, it is assumed that each meal event is far enough apart in time such that the initial change in glucose rise and the subsequent reversal is discernible from signal artifacts that may exist in the available sensor data measurements. Furthermore, the interaction between a meal and insulin is such that insulin cannot perfectly cancel the change in glucose due to the meal. This excludes clamps where both dextrose and insulin are intravenously administered at near constant infusion rates. Also, a meal may have insufficient insulin bolus, which would result in a higher post prandial glucose level than the pre-prandial glucose level, and may still continue to rise afterwards. In other words, the glucose response looks like the superposition of an increasing ramp and a textbook meal response (i.e. starting at a certain value, rising rapidly at the start of the meal, and decreasing at a slightly slower rate than the rate of increase). Accordingly, in certain embodiments, the glucose data are screened for local acceleration optima (ie. minima or maxima) as the initial candidate, and further refines the candidate start and peak instances by eliminating false candidates and adjusting the position of the final set of candidates.

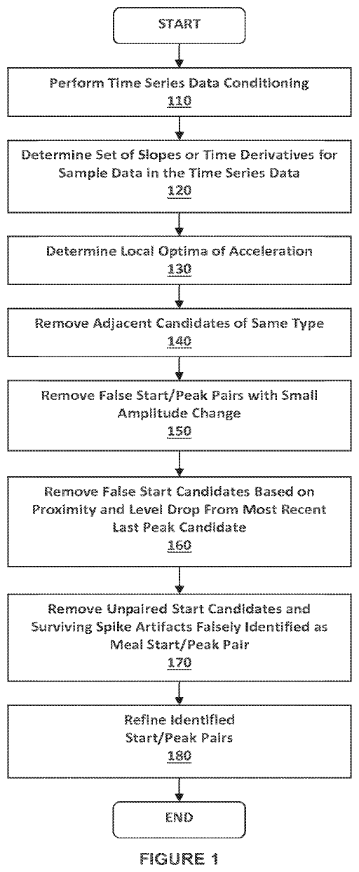

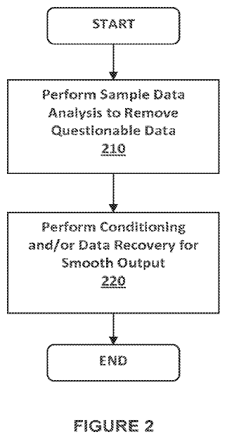

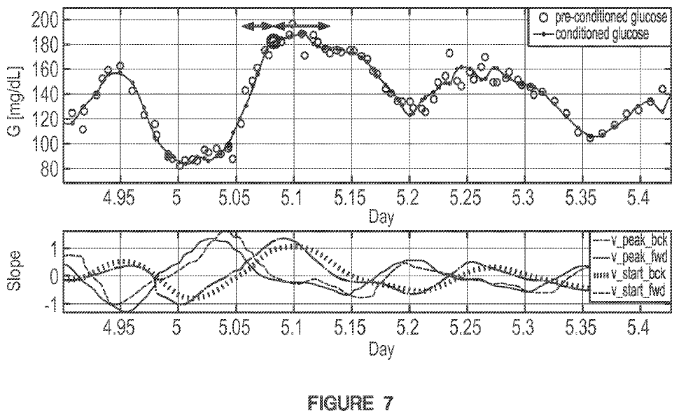

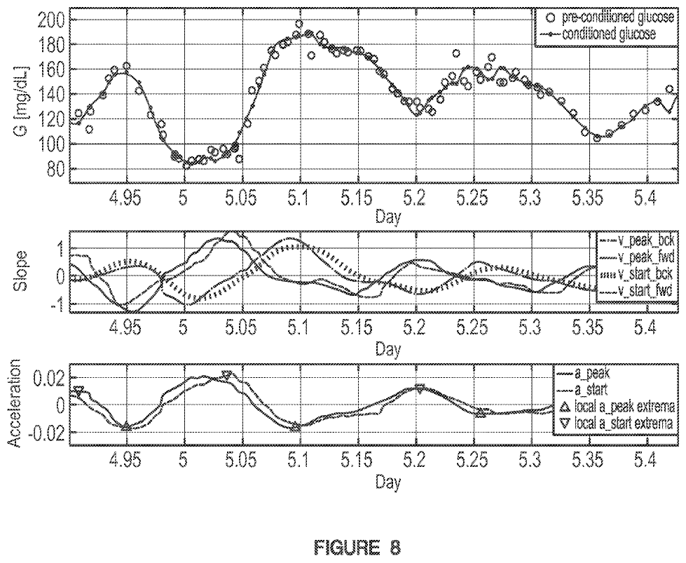

FIG. 1 illustrates a flowchart for meal start and peak detection routine in accordance with certain embodiments of the present disclosure. Referring to FIG. 1, sampled analyte (e.g. glucose) data from analyte monitoring systems or devices are collected or received and time series data conditioning is performed (110) that includes, for example, data conditioning to remove questionable readings from the sampled data and smoothing out the final result as described in detail below in conjunction with FIGS. 2-4. In certain embodiments, the data conditioning results in generating regularly spaced glucose values from irregularly sampled data. In certain embodiments, data conditioning includes determining whether sampled glucose data may be outliers when compared to sampled glucose data that are temporally in close proximity with each other. FIG. 6 illustrates data conditioning and/or data recovery for smooth output in conjunction with the routines above in certain embodiments of the present disclosure.

Referring to FIG. 1, after performing time series data conditioning (110), for each sampled sensor data, a set of slopes for the sampled analyte data are determined (120). That is, for each sampled data, a set of time derivatives in each time instance k of the glucose time series is determined (120). Separate sets of slopes or time derivatives are calculated to determine peak and meal start candidates. The selection of the time window duration where these sets of time derivatives are to be determined from, in the order of a few hours, are tuned to detect meal responses and ignore transients and other unrelated elements of glucose time series progression. The number of sampled data involved depends on the relative timestamps associated with when the sampled data was acquired.

More specifically, in certain embodiments, the set of slopes for determining a peak candidate, is a pair of slopes; one generated by computing rate of change in a forward window, and another generated by computing rate of change in a backward time window. Specifically, in certain embodiments, determining the forward and backward time window rates of change for the peak candidate includes using the sampled glucose data that are in the forward time window (i.e. from the present measurement at k to its near future time instance, such as 2˜3 hours later) for the peak candidate, and then fit a straight line using Least-Squares error (LS) fit method. The slope is the forward rate of change for the peak candidate, v_peak_fwd(k). Further, determining the backward window rate includes using sampled glucose data that are in the backward window (i.e. from the present measurement at k to its near past time instance, such as 1˜2 hours prior) for the peak candidate, and then fit a straight line using Least-Squares error (LS) fit method. The slope is the backward rate of change for the peak candidate, v_peak_bck(k).

In addition, the set of slopes for determining a meal start candidate is a pair of slopes; one generated by computing rate of change in a forward window, and another generated by computing rate of change in a backward time window. More specifically, in certain embodiments, determining forward and backward window rates of change for the meal start candidate includes using the sampled glucose data that are in the forward window (i.e. from the present measurement at k to its near future time instance, such as 1˜1.5 hours later) for the meal start candidate, and then fit a straight line using Least-Squares error (LS) fit method. The slope is the forward rate of change for the meal start candidate, v_start_fwd(k). In certain embodiments, the forward window for peak and meal start candidates does not necessarily have the same width. Further, determining the backward window rate includes using sampled glucose data that are in the backward window (i.e. from the present measurement at k to its near past time instance, such as 2˜3 hours prior) for the meal start candidate, and then fit a straight line using Least-Squares error (LS) fit method. The slope is the backward rate of change for the meal start candidate, v_start_bck(k). In certain embodiments, the backward window for peak and meal start candidates does not necessarily have the same width.

Referring to FIG. 1, the slope determination or determination of time derivatives for sampled data in the time series data (120) in certain embodiments includes determining the acceleration for the peak candidate, a_peak(k), where the acceleration for the peak candidate, a_peak(k), is defined as (v_peak_fwd(k)−v_peak_bck(k))/T_peak, where T_peak is a pre-determined sample period scaling factor for the peak candidate determination (for example, 1˜3 hours).

Further, the slope determination or determination of time derivatives for sampled data in the time series data (120) further includes determining the acceleration for the start candidate, a_start(k), where acceleration for the start candidate, a_start(k) is defined as (v_start_fwd(k)−v_start_bck(k))/T_start, where T_start is a pre-determined sample period scaling factor for the meal start candidate determination (for example, 1˜3 hours).

In this manner, in certain embodiments, the determination of time derivatives for sampled data in the time series data (120) includes slope or rate of change determination for each instance k of the sampled data, as shown for example, in FIG. 7. More specifically, FIG. 7 illustrates determination of backward and forward slopes for peak and meal start candidates in conjunction with the routines above in certain embodiments of the present disclosure. Referring to FIG. 7, circled sampled glucose data measurement instance and the nearby arrows illustrate the approximate size of the forward (to the right of the measurement instance) and backward (to the left of the measurement instance) time windows for the slope determinations.

Referring back to FIG. 1, after the determination of time derivatives for sampled data in the time series data (120), the local optima of acceleration is determined (130). More specifically, in certain embodiments, the local optima of acceleration are identified based upon signal analysis to identify extreme bend points. More specifically, in certain embodiments, at each time instance k, the determined acceleration for the peak candidate, a_peak, that falls within the forward window (incorporating data from present to 1˜2 hrs later) for the peak candidate is determined, with the exception of the value at time instance k, a_peak(k). Further, at each time instance k, value a_peak that falls within the backward window (incorporating data from 1˜2 hrs before to the present) for the peak candidate is determined, with the exception of the value at time instance k, a_peak(k). If the value a peak(k) is less than or equal to the minimum a_peak values in the two aforementioned windows, the current time instance k is determined as a peak candidate during the determination of local optima of acceleration (130).

At each time instance k, the determined acceleration for the meal start candidate, a_start, that falls within the forward window (incorporating data from present to 1˜2 hrs later) for the meal start candidate is determined, with the exception of the value at time instance k, a_start(k). At each time instance k, value a_start that falls within the backward window (incorporating data from 1˜2 hrs before to the present) for the meal start candidate is determined, with the exception of the value at time instance k, a_start(k). If the value a_start(k) is greater than or equal to the maximum a_start values in the two aforementioned windows, then the current time instance k is determined as a meal start candidate during the determination of local optima of acceleration (130). In certain embodiments, if a time instance k has been previously identified as a peak candidate, and is also identified as a meal start candidate, the meal start candidate tag is moved to the next instance k+1. FIG. 8 illustrates determination of acceleration and the identification of local acceleration optima in conjunction with the routines above in certain embodiments of the present disclosure. More specifically, FIG. 8 illustrates the identification of the peak and meal start candidates described above and as marked by up and down triangles on the acceleration plot of FIG. 8.

More specifically, in certain embodiments, from all instances k of sampled glucose data in a time series, an initial subset of data is generated that includes all instances, m, identified or tagged as either a peak or meal start candidate from local optima of acceleration determination (130). For example, from each sampled glucose measurement instance k=1, 2, 3, . . . 10000, of 10000 measurement points, 5 candidates are identified from instances k=100, 150, 300, 400, and 700. The 5 candidate instances m=1, 2, 3, 4, 5, would be associated with the original instances as follows: the first candidate instance m=1 corresponds to the original instance at k=100, and the 2^{nd} candidate instance m=2 corresponds to the original instance at k=150, etc.

Referring back to FIG. 1, after the determination of local optima of acceleration (130), data analysis continues to identify and remove false meal start and peak candidates. In a first stage of analysis and removal, adjacent candidates of the same type are removed (140). That is, since a meal start event cannot be adjacent in time to another meal start event, and similarly, a peak meal response event cannot be adjacent in time to another peak meal response event, during the first stage of analysis and removal, adjacent candidates of the same type are identified and removed from the data set under consideration.

More specifically, from the initial subset of data including all instances, m, a first stage list of peak and meal start candidates identified as adjacent candidates of the same type is generated. From this first stage list, peak candidates are removed because the next instance of an adjacent peak candidate has a larger glucose value. That is, a peak candidate is removed during the first stage based on the following criteria: (1) the next instance m+1 in the subset is also a peak candidate; (2) the next instance m+1 in the initial subset has a larger glucose value than the current instance m; and (3) the rate from the forward peak calculation of the current instance m, v_peak_fwd(m), is more than a non-negative noise floor v_min_rise (e.g. 0.5 mg/dL/min). Calculated rates of change whose absolute numbers are close to zero tend to contain a lot of noise. Additionally, in certain embodiments, a peak candidate is also removed during the first stage if the prior instance of an adjacent peak candidate has a larger glucose value, i.e., based on the following criteria: (1) the previous instance m−1 in the initial subset is also a peak candidate; and (2) previous instance m−1 in the initial subset has a larger glucose value than the current instance m.

Furthermore, in certain embodiments, from the first stage list, meal start candidates are removed because the previous instance of an adjacent meal start candidate has a smaller glucose value. That is, a meal start candidate is removed during the first stage based on the following criteria: (1) the previous instance m−1 in the initial subset is also a meal start candidate; (2) the previous instance m−1 in the initial subset has a smaller glucose value than the current instance m; and (3) the value a_start(m−1) is smaller than a_start(m). In addition, in certain embodiments, a meal start candidate is also removed during the first stage if the next instance of an adjacent meal start candidate has an equal or smaller glucose value, i.e., based on the following criteria: (1) the next instance m+1 in the initial subset is also a meal start candidate; and (2) the next instance m+1 has a glucose value that is either equal to or less than the glucose value than the current instance m.

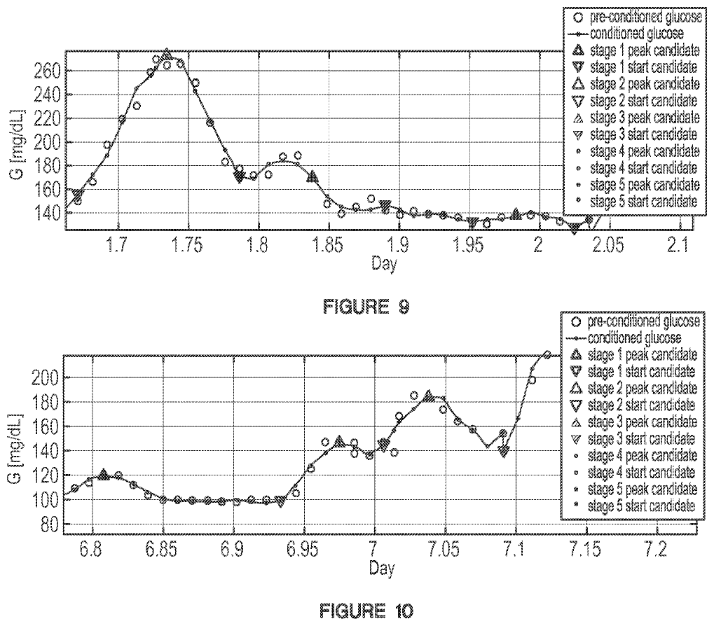

In this manner, in certain embodiments, adjacent meal start candidate or peak candidates of the same type are identified and removed from the data set under consideration. FIG. 9 illustrates an example of removal of adjacent candidates of the same type in conjunction with the routines above in certain embodiments of the present disclosure. More specifically, FIG. 9 is an example illustration of a meal start candidate at around 1.9 days that was identified during the local optima of acceleration determination (130), but was removed during the first stage of analysis and removal based on analysis determining the meal start candidate as adjacent candidate of the same type (140).

Referring again to FIG. 1, after removing adjacent candidates of the same type (140), the routine continues with a second stage of analysis and removal to identify and remove false meal start/peak pairs with small amplitude change (150). More specifically, in certain embodiments, an analysis is performed on the subset of remaining instances of peak candidates and meal start candidates following the first stage of removal based on adjacent candidates of the same type, i.e., a first stage subset. During the second stage, every peak candidate in the first stage subset is analyzed to determine whether the change in glucose value from the previous instance m−1, which would be a meal start candidate, to the current peak candidate m is sufficiently large. In other words, the current peak candidate m is removed from the first stage subset of tagged start or peak candidates when the following criteria are met: (1) previous instance m−1 in the first stage subset (after removal of adjacent candidates of the same type) is tagged as a meal start candidate; (2) the current instance m in the first stage subset is tagged as a peak candidate; and (3) the difference between the amplitude of the current instance m and the previous instance m−1 is less than or equal to g_min_amplitude, i.e. g(m)−g(m−1)<=g_min_amplitude, wherein g is the instance amplitude or level of glucose. Moreover, in certain embodiments, when a peak candidate is removed under these conditions, the corresponding meal start candidate, that is the previous instance m−1, is also removed.

By way of an example, FIG. 9 illustrates examples of removal of false meal start and peak candidate pairs with a small amplitude change in conjunction with the routines above in certain embodiments of the present disclosure. More specifically, FIG. 9 illustrates 2 pairs (around 1.8 days and 1.95 days) that were removed based on the analysis described herein to remove false meal start/peak pairs with small amplitude change.

Referring still to FIG. 1, after identifying and removing false meal start/peak pairs with small amplitude change (150), the routine continues with a third stage of analysis and removal to identify and remove false meal start candidates based on proximity and level drop from the most recent last peak candidate (160). That is, in certain embodiments, meal start candidates that are too close in time to a prior peak candidate and whose glucose value is not significantly lower than the glucose value of its prior peak candidate, are removed from the subset of remaining instances of peak candidates and meal start candidates following the second stage of removal, i.e., a second stage subset.

More specifically, in certain embodiments, during the third stage, it is determined whether the position of each meal start candidate with respect to a previous peak candidate is reasonable. That is, a meal start candidate at instance m is removed when the following criteria are met: (1) the previous instance m−1 in the second stage subset (after removal of start/peak pair with small amplitude change) is tagged as a peak candidate (e.g. see up triangle at around 6.975 days in FIG. 10); (2) the current instance m in the second stage subset is identified or tagged as a meal start candidate (e.g. see down triangle at around 7 days in FIG. 10); (3) the next instance m+1 in the second stage subset is identified or tagged as a peak candidate (e.g. see up triangle at around 7.04 days in FIG. 10); (4) the average value of v_start_bck(m) (see down triangle at around 7 days of FIG. 10) and v_peak_fwd(m−1) (see up triangle at around 6.975 days of FIG. 10) is greater than a maximum post-prandial recovery descent rate, v_max_descent (e.g. ¼ mg/dL/min); and (5) the difference between the glucose value of the current instance m and the previous instance m−1, g(m)−g(m−1), is less than or equal to a minimum required drop from a previous peak, g_min_drop (e.g. 5˜10 mg/dL). Moreover, when these criteria are met and a meal start candidate is removed, the peak candidate at the previous instance m−1, is also removed. FIG. 10 illustrates a meal start candidate at around 7 days that was removed, along with the prior peak candidate, due to proximity and level drop.

Referring again to FIG. 1, after removing meal start candidates based on proximity and level drop from the most recent last peak candidate (160), the routine continues, in certain embodiments, with a fourth stage of analysis and removal to identify and remove unpaired meal start candidates and surviving spike artifacts falsely identified as meal start/peak pairs (170). Surviving spike artifacts might happen if Time Series Data Conditioning (110) does not completely remove all artifacts. More specifically, in certain embodiments, surviving spike artifacts falsely identified as meal start/peak pairs, are removed from the subset of remaining instances of peak candidates and meal start candidates following the third stage of removal, i.e., a third stage subset. For each instance m in the third stage subset that is a start candidate, those whose next instance m+1 is not a peak candidate is removed. That is, a current meal start candidate at instance m is removed from the third stage subset if all of the following applies: (1) the current instance m is tagged as a meal start candidate; (2) the next instance m+1 is tagged as a peak candidate; and (3) the aggregate glucose rate of change, as calculated from g(m+1)−g(m), divided by the time interval between the two instances m+1 and m, is larger than a maximum allowable initial post-prandial rate of change, v_max_initialSpike (e.g. 6 mg/dL/min, which is a rate of change that is likely not sustainable between two candidate points).

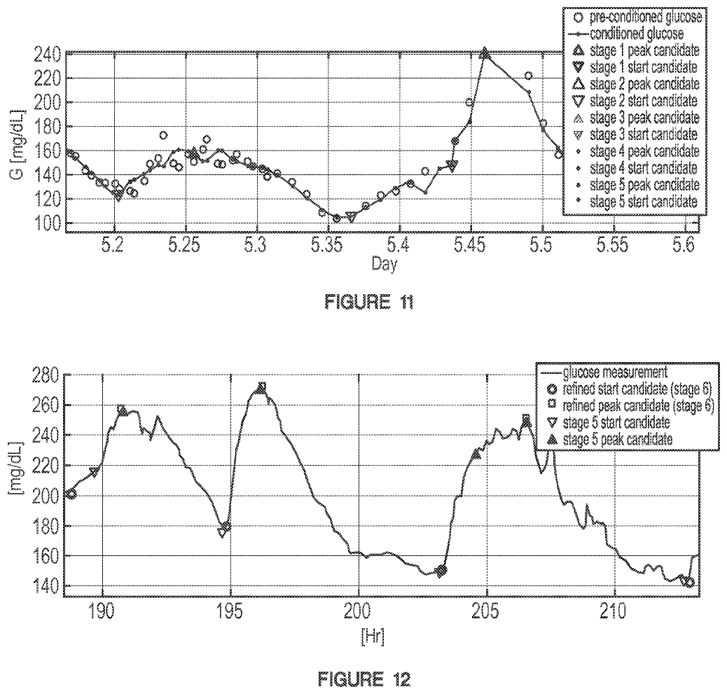

Referring to the Figures, FIG. 11 illustrates removal of unpaired meal start candidates and surviving spike artifacts falsely identified as a meal start/peak pair in conjunction with the routines above in certain embodiments of the present disclosure. An example of a start candidate to be removed by this criteria is shown in FIG. 11, at around 5.35 days, where the next instance at around 5.44 days is also a start candidate.

In certain embodiments, because of the asymmetrical forward and backward time windows used to determine the pair v_start_fwd and v_start_bck, as well as the pair v_peak_fwd and v_peak_bck, and since a post-prandial meal response may be followed by a subsequent post-prandial meal response without sufficient time for the original post-prandial meal response to revert to the baseline or fasting glucose levels, the identification of meal start and peak candidate may be visibly biased slightly before or after the likely instance. Accordingly, in certain embodiments, these likely instances are analyzed and adjusted as discussed below.

More specifically, after the four stages of analysis and removal discussed above are complete, the remaining identified meal start and peak candidates are refined (180) in certain embodiments. That is, for each sampled glucose data time instance k, a simple forward and backward slope is determined. For example, all sampled glucose data measurement instances k are evaluated to refine the meal start and peak candidates remaining after the four stages of analysis and removal, identified in the subset of instances m. In certain embodiments, the time window sizes used in determining v_peak_fwd, v_peak_bck, v_start_fwd, v_start_bck, may be larger and asymmetric compared to the determinations steps that follow determining v_peak_fwd, v_peak_bck, v_start_fwd, v_start_bck. In this manner, false candidates due to signal artifacts are rejected earlier on in the routine as described in conjunction with FIG. 1, and by the start of the routine to refine the identified meal start and peak instances (180), the candidates are sufficiently localized to the true meal start and peak. Further, the smaller time windows provide a better precision in the determination.

In particular, for each sampled glucose data at instance k, g(k), an available sample that is as close to 30 minutes prior to k as possible, g_prev(k) is identified. Also, for each sampled glucose data at instance k, g(k), an available sample that is as close to 30 minutes after k as possible, g_after(k) is identified. Then, forward and backward slopes, v_fwd(k) and v_bck(k). v_fwd(k) are determined by taking the difference g_after(k)−g(k), and dividing it by their time interval (around 30 minutes). Also, backward slope v_bck(k) is calculated by taking the difference g(k)−g_prev(k), and dividing it by their time interval. The difference in slope, dv(k), is determined by taking the difference v_fwd(k)−v_bck(k).

For every instance k where a meal start or peak has been identified during removal of start candidates based on proximity and level drop analysis from most recent last peak candidate (160), the time instances in pairs of meal start and peak are identified.

For each identified meal start/peak pair a glucose time series, g_array_start, up to 90 minutes prior to the identified start candidate, and up to 60 minutes after the identified start candidate is defined. The defined glucose time series, g_array_start includes the meal start candidate that survived the data processing (110 to 170) in the routine described above in conjunction with FIG. 1. Also, a glucose time series, g_array_peak, up to 60 minutes prior to the identified peak candidate, and up to 180 minutes after the identified peak candidate is defined. The glucose time series, g_array_peak includes the peak candidate that survived the data processing (110 to 170) in the routine described above in conjunction with FIG. 1. Then, g_array_peak from any sampled glucose data are trimmed whose timestamp overlaps the start time of the next pair in the routine where the unpaired start candidates and surviving spike artifacts falsely identified as meal start/peak pair are removed (170). For each value in g_array_start and g_array_peak, the corresponding difference in slope values, dv are determined from the same instances. Arrays of these values, dv_array_start and dv_array_peak are defined.

Thereafter, in certain embodiments, a subset of time instances are determined such that (1) measured glucose value at these instances are greater than or equal to the 75^{th} percentile of g_array_peak, and (2) dv value at these instances are less than or equal to the 25^{th} percentile of dv_array_peak. If such a subset contains data, then the highest glucose value in this subset, g_max, and its corresponding instance, is stored. Furthermore, the routine determines a subset of time instances such that (1) measured glucose value at these instances are less than or equal to the 25.sup.th percentile of g_array_start, and (2) dv value at these instances are greater than or equal to the 75.sup.th percentile of dv_array_start. If such a subset contains data, then the lowest glucose value in this subset, g_min, and its corresponding instance, is stored. Then the peak and start candidate for this pair with the highest glucose value in the subset, g_max and the lowest glucose value in the subset, g_min, are updated based on the following criteria: (1) the lowest glucose value in the subset, g_min, and the highest glucose value in the subset, g_max, exist and are finite; (2) the instance of lowest glucose value in the subset, g_min, occurs prior to the instance of the highest glucose value in the subset, g_max; and (3) the lowest glucose value in the subset, g_min, is less than the highest glucose value in the subset, g_max.

FIG. 12 illustrates refinement of identified meal start and peak instances in conjunction with the routines above in certain embodiments of the present disclosure. More specifically, FIG. 12 provides an example illustration of the effect of the routine to refine the identified start/peak pairs (180) of FIG. 1 when glucose measurement is sampled at a relatively fast sample period of once every minute. For sparser sample periods (such as illustrated in FIG. 11), the number of sampled glucose data that can be a viable peak or meal start candidates are much smaller than faster sample periods. As a result, the refinement of identified meal start/peak pairs (180) is more useful in certain embodiments, around time periods with a lot of measurements than periods with sparse measurements.

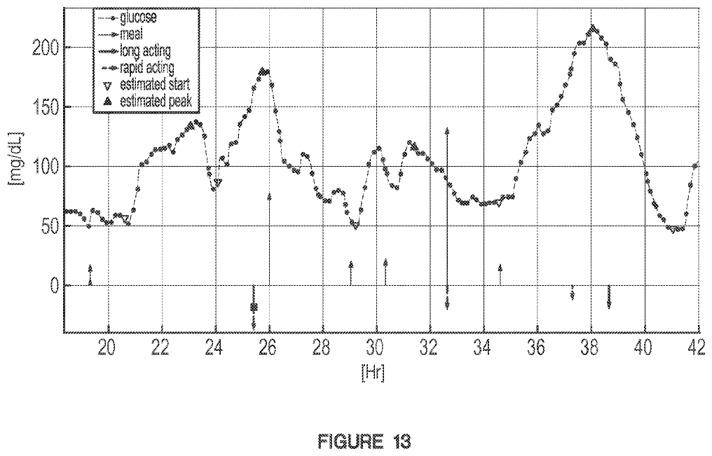

FIG. 13 illustrates an example of comparison of estimated meal start determination in conjunction with the routines described herein against manually marked meal start events. Referring to FIG. 13, there is shown sampled glucose data from a patient, along with patient-recorded meal marker, long acting insulin, and rapid acting insulin. The estimated meal start and peak as described in conjunction with FIG. 1 above is also shown. The plot in FIG. 13 covers approximately one day, starting from a fasting period (up to around 21 hours since glucose sensor start (to acquire sampled glucose data)), followed by a series of meals, and a potentially unrecorded rescue carbohydrate at around hour 41. There are 7 meal markers recorded, two of them within a few minutes at around hour 19. It can be seen from FIG. 13 that the first two meal markers appear to correspond to the increase estimated at around hour 21. The third meal marker may be a late entry from the lunch at hour 24, and the subsequent two entries may be snacks. The two snacks were assumed as a single meal by the estimation routine in accordance with the embodiments of the present disclosure, due to an assumption about minimum duration of meals reflected in the duration of the forward and backward windows of the peak and start candidates during the local optima of acceleration determination. The last two may correspond to the bulk of dinner and a dessert, although the glucose response seems to be delayed by about 3 hours.

Referring back to the Figures, FIG. 2 illustrates a flowchart for performing time series sampled analyte data conditioning of the meal start and peak detection routine of FIG. 1 in accordance with certain embodiments of the present disclosure. Referring to FIGS. 1 and 2, performing time series sampled analyte data conditioning of the meal start and peak detection (110) in certain embodiments includes performing sampled data analysis to remove questionable data, where physiological limits are used to compare each sampled glucose data in the context of other temporally closely located sampled glucose data (210). Thereafter, data conditioning and/or recovery is performed to smooth the data output (220), where surviving sampled data are conditioned to minimize noise, and removed measurements are supplemented by sampled data based on other temporally closely located sampled data.

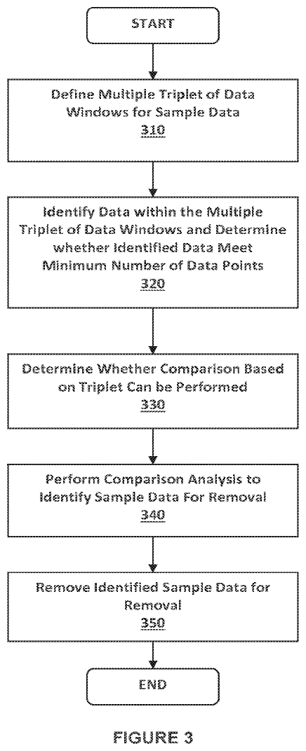

FIG. 3 illustrates a flowchart for sampled data analysis to remove questionable data of FIG. 2 in accordance with certain embodiments of the present disclosure. Referring to FIG. 3, in certain embodiments, removal of questionable sampled glucose data includes data processing and analysis as described below. More specifically, for each sampled data instance, more than one triplet of time windows is defined to address data stream with a range of sample time intervals. That is, a first triplet of left, center, and right time windows ScreenLeft1, ScreenCenter1, and ScreenRight1, respectively, are defined where (1) the left time window only looks at available measurements prior to the current instance (e.g. from 30 minutes ago to 3 minutes ago); (2) the right time window only looks at available measurements after the current instance (e.g. from 3 minutes to 30 minutes after the current instance); (3) the center time window only looks at available measurements slightly before the current instance and slightly after the current instance (e.g. within ±3 minutes of the current instance); and (4) each time window requires a minimum number of available points (e.g. 1 for the center time window, 2 for the left time window, and 2 for the right time window).

Furthermore, a second triplet of left, center, and right time windows ScreenLeft2, ScreenCenter2, and ScreenRight2, respectively are defined, where (1) left time window is narrower than that of ScreenLeft1 (e.g. from 15 minutes ago to 3 minutes ago), but requires a larger number of minimum available points (e.g. 6 points); (2) right time window is narrower than that of ScreenRight1 (e.g. from 3 minutes to 15 minutes after the current instance), but requires a larger number of minimum available points (e.g. 6 points); and (3) center time window requires a larger number of minimum available points (e.g. 4 points). Also, a maximum allowable range ScreenMaxRange and maximum allowable relative range ScreenMaxRelativeRange are defined to be used to compare multiple estimates based on the different time windows.

Referring back to FIG. 3, after defining multiple triplets of data windows for the sampled data (310), data within the multiple triplets of data windows are identified and it is determined whether the identified data meet the minimum number of data points (320). More specifically, for each sampled glucose data instance, measurements that fall within the multiple triplets of windows as set forth above are identified, and it is determined whether or not the number of available points in each time window meets the respective minimum number of points. Then, it is determined whether comparison based on each triplet can be performed (330) based on the following criteria: (1) comparison within the first triplet can be performed when there is sufficient number of measurements in ScreenCenter1, and either there is sufficient number of sampled data in ScreenLeft1 or ScreenRight1; and (2) comparison within the second triplet can be performed when there is sufficient number of measurements in ScreenCenter2, and either there is sufficient number of measurements in ScreenLeft2 or ScreenRight2.

Furthermore, for each sampled glucose data instance, if comparison within the first triplet can be performed the following routines are performed (340). More specifically, yCenter1, an estimate of current measurement instance based on ScreenCenter1, is determined by taking the average of available points in ScreenCenter1, yRight1, an estimate of current measurement instance based on ScreenRight1, is determined by performing a least-square error fit of a straight line using available points in ScreenRight1, evaluated at the instance of the current sampled data. The estimate of current measurement instance based on ScreenRight1, yRight1, is not determined if the number of points in ScreenRight1 is insufficient. Also, yLeft1, an estimate of current measurement instance based on ScreenLeft1, is determined by performing a least-square error fit of a straight line using available points in ScreenLeft1, evaluated at the instance of the current measurement. The estimate of current measurement instance, yLeft1, is not determined if the number of points in ScreenLeft1 is insufficient.

If comparison within the second triplet can be performed, yCenter2, an estimate of current measurement instance based on ScreenCenter2, is determined by performing a least-square error fit of a straight line using available points in ScreenCenter2, evaluated at the instance of the current measurement. The estimate of current measurement instance based on ScreenCenter2, yCenter2 is not determined if the number of points in ScreenCenter2 is insufficient. Also, yRight2, an estimate of current measurement instance based on ScreenRight2, is determined by performing a least-square error fit of a straight line using available points in ScreenRight2, evaluated at the instance of the current measurement. The estimate of current measurement instance based on ScreenRight2, yRight2 is not determined if the number of points in ScreenRight2 is insufficient. Additionally, yLeft2, an estimate of current measurement instance based on ScreenLeft2, is determined by performing a least-square error fit of a straight line using available points in ScreenLeft2, evaluated at the instance of the current measurement. The estimate of current measurement instance based on ScreenLeft2, yLeft2 is not determined if the number of points in ScreenLeft2 is insufficient. Then, estimates of the current measurement instance based on the first triplet, yCenter1, yRight1, and yLeft1, are updated by estimates based on the second triplet (e.g. assign the value of yCenter2 to yCenter1, assign the value of yRight2 to yRight1, and assign yLeft2 to yLeft1), if the determination is available.

In addition, for each sampled data instance, if comparison within the first triplet can be performed, available yCenter1, yLeft1, and yRight1 measurements are collected, and the following values are determined: (1) yAvg, the average of the available values, (2) yMin, the smallest of the available values, (3) yMax, the largest of the available values, (4) yRange, the absolute value of the difference between yMin and yMax, and (5) yRelativeRange, the value of yRange divided by yAvg. Then, the values yRelativeRange and yRange are compared against the thresholds ScreenMaxRelativeRange and ScreenMaxRange, respectively. If either one exceeds the threshold, the current sampled glucose data instance is identified for removal. In certain embodiments, identifying for removal of any sampled glucose data instance is not performed until all sampled glucose data instances have been evaluated.

On the other hand, for each sampled glucose data instance, if the comparison within the first triplet cannot be performed (340), the sampled glucose data instance is not identified for removal. Thereafter, sampled glucose data instances identified for removal are removed from the data set under analysis (350).

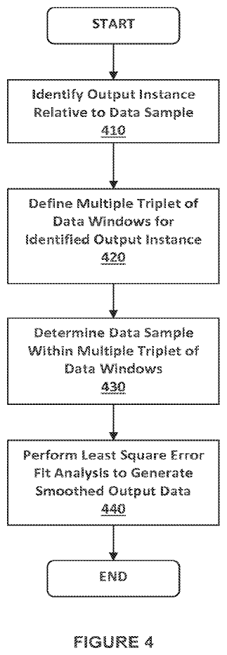

FIG. 4 illustrates a flowchart for data conditioning and/or data recovery for smooth output of FIG. 2 in accordance with certain embodiments of the present disclosure. Referring to FIGS. 2 and 4, in certain embodiments, data conditioning and/or recovery performed to smooth the data output (220) (FIG. 2) includes identifying output instance relative to data sample (410). That is, instances where output is desired is defined by, for example, (1) defining output instances as instances where the original sampled glucose data are found in which case, the output instances will take on the same timestamps as the original data, (2) defining output instances as instances where the original sampled glucose data are found, but were not marked for removal at step 210 (FIG. 2), or (3) defining output instances by a new arbitrary, but regular, sample interval (e.g. once every 8 minutes, or once every 30 minutes).

Referring back to FIG. 4, after identifying output instance relative to data sample (410), multiple triplet of data time windows for identified output instance is defined (420). More particularly, in certain embodiments, for each identified output instance, more than one triplet of time windows are defined to process data streams with a range of data sample time intervals. Specifically, in certain embodiments, a first triplet of left, center, and right windows SmoothLeft1, SmoothCenter1, and SmoothRight1, respectively, are defined where (1) left window, SmoothLeft1 only looks at available measurements prior to the current instance (e.g. from 50 minutes ago to 5 minutes ago); (2) right window, SmoothRight1 only looks at available measurements after the current instance (e.g. from 5 minutes to 50 minutes after the current instance); and (3) center window, SmoothCenter1 only looks at available measurements before the current instance and after the current instance (e.g. within ±32 minutes of the current instance). In certain embodiments, each window requires a minimum number of available points (e.g. 2 for the center window, 3 for the left window, and 3 for the right window).

Furthermore, in certain embodiments, for each identified output instance, more than one triplet of time windows are defined to process data streams with a range of data sample time intervals by defining a second triplet of left, center, and right windows SmoothLeft2, SmoothCenter2, and SmoothRight2, where (1) left window is narrower than that of SmoothLeft1 (e.g. from 20 minutes ago to 5 minutes ago), but requires a larger number of minimum available points (e.g. 9 points); (2) right window is narrower than that of SmoothRight1 (e.g. from 5 minutes to 20 minutes after the current instance), but requires a larger number of minimum available points (e.g. 9 points), and (3) center window is narrower than that of SmoothCenter1 (e.g. from 7 minutes prior to 7 minutes after the current instance), but requires a larger number of minimum available points (e.g. 9 points).

Referring again to FIG. 4, after defining multiple triplet of data time windows for identified output instance (420), for each output instance, sampled glucose data that fall within the defined multiple triplets of time windows are identified (430). It is also determined whether the number of available sampled glucose data points in each time window meets the respective minimum number of points.

Thereafter, Least Square error fit analysis is performed to generate smoothed output data (440). For example, in certain embodiments, ySmoothCenter1, an estimate of current output instance based on SmoothCenter1, is determined by performing a least-square error fit of a straight line using available points in SmoothCenter1, evaluated at the current output instance. The estimate of current output instance based on SmoothCenter1, ySmoothCenter1 is not determined if the number of points in this window is insufficient. Also, ySmoothRight1, an estimate of current output instance based on SmoothRight1, is determined by performing a least-square error fit of a straight line using available points in SmoothRight1, evaluated at the current output instance. The estimate of current output instance based on SmoothRight1, ySmoothRight1 is not determined if the number of points in this window is insufficient. In addition, ySmoothLeft1, an estimate of current output instance based on SmoothLeft1, is determined by performing a least-square error fit of a straight line using available points in SmoothLeft1, evaluated at the current output instance. The estimate of current output instance based on SmoothLeft1, ySmoothLeft1 is not determined if the number of points in this window is insufficient. Moreover, ySmoothCenter2, an estimate of current output instance based on SmoothCenter2, is determined by performing a least-square error fit of a straight line using available points in SmoothCenter2, evaluated at the current output instance. Similarly, the estimate of current output instance based on SmoothCenter2, ySmoothCenter2 is not determined if the number of points in this window is insufficient. Otherwise, ySmoothCenter1 is updated by assigning the value of ySmoothCenter2 to ySmoothCenter1. Further, ySmoothRight2, an estimate of current output instance based on SmoothRight1, is determined by performing a least-square error fit of a straight line using available points in SmoothRight2, evaluated at the current output instance. Again, the estimate of current output instance based on SmoothRight1, ySmoothRight2 is not determined if the number of points in this window is insufficient. Otherwise, ySmoothRight1 is updated by assigning the value of ySmoothRight2 to ySmoothRight1. Still further, ySmoothLeft2, an estimate of current output instance based on SmoothLeft2, is determined by performing a least-square error fit of a straight line using available points in SmoothLeft2, evaluated at the current output instance. The estimate of current output instance based on SmoothLeft2, ySmoothLeft2 is not determined if the number of points in this window is insufficient. Otherwise, ySmoothLeft1 is updated by assigning the value of ySmoothLeft2 to ySmoothLeft1.

Thereafter, ySmoothAvgSide, the average of available ySmoothRight1 and ySmoothLeft1 is determined. If both ySmoothCenter1 and ySmoothAvgSide can be determined, ySmooth, the smoothed, final output for this output instance is determined, by assigning ySmooth as the average of ySmoothCenter1 and ySmoothAvgSide.

In the manner described above, in certain embodiments, the meal start and peak estimation routine includes performing sample data analysis to remove questionable data (210) and then performing data conditioning and/or data recovery for smooth output (220) to perform time series data conditioning (110) before the time derivatives for sample data in the time series data are determined (120).

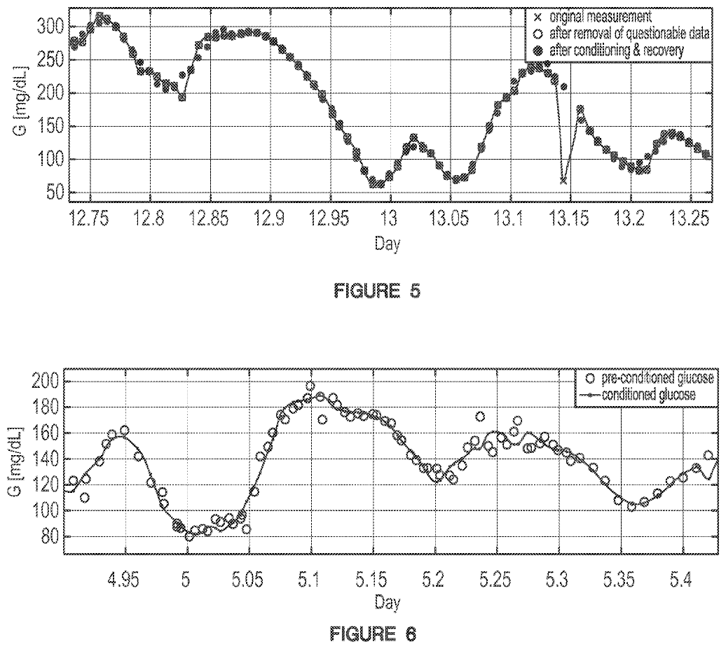

FIG. 5 illustrates sample data analysis to remove questionable data and performing condition and/or data recovery for smooth output in conjunction with the routines above in certain embodiments of the present disclosure. As shown, sampled glucose data (x) are processed to screen out questionable data. After questionable data are removed, the dataset (circle) goes through the conditioning process described above to obtain the final output values (dots). In this example, the output instances are identical to the measurement instances.

In the manner described above, in certain embodiments of the present disclosure, meal start events and peak events are estimated or determined based on analysis of time series of sampled glucose data from, for example, an in vivo glucose sensor that generates signals corresponding to the monitored glucose level at a specific or programmed or programmable time intervals and which signals can be further processed and analyzed in the manner described above, to estimate meal start and peak events.

In certain embodiments, meal marker manually entered by the user is compared against the estimated meal start determined in accordance with the embodiments of the present disclosure based on sampled glucose data that includes real time data and historical data. A short elapsed time after the meal marker is entered, when the glucose measurements are sufficient to generate a nearby estimate, the user may be prompted (using an analyte monitoring device user interface, for example) to adjust the meal marker timestamp to the estimated instance. In this exemplary embodiment, no estimated meal start replaces user entered marker unless confirmed by the user.

In certain embodiments, retrospective and pseudo-retrospective analysis of time spaced sampled glucose data are performed to generate user viewable reports or analysis results associated with the meal start and peak meal response events estimation, and which are viewable on the user interface of a hand-held data communication device, a mobile telephone screen, a smart phone user interface, or computing device, where the analysis is performed based on collected glucose data acquired up to the current time.

In certain embodiments, data reports are generated based on the meal start event and/or peak meal response events estimated in accordance with the present disclosure, to replace, supplement, revise or confirm such reports that rely on either a) meal tags made by users, b) meal bolus indications from bolus calculators, insulin pumps or smart insulin injection systems, or c) fixed meal times.

In certain embodiments, the meal start event or peak meal response estimation routine in accordance with the present disclosure is used to either cross-check or confirm the absence of presence of meal tags manually entered by the user or a healthcare provider.

In certain embodiments, the meal start event or peak meal response estimation routine in accordance with the present disclosure is used in conjunction with a report or table that is generated from glucose data which is separated into 5 different time-of-day bins defined by fixed meal times and bedtime. The bins may be determined by meal start events based on the meal start event or peak meal response estimation routine in accordance with the present disclosure with predetermined categorization parameters, such as, for example, categorizing identified meal times as a particular meal. For instance, an estimated meal start event would be defined as breakfast if it occurred between 4 am and 10 am.

In certain embodiments, other report designs are contemplated. One example is a report that is used to determine fasting glucose level for diagnosing diabetes. The report algorithm in certain embodiments determine all of the breakfast start times and use a glucose value some time prior to these start values to generate a statistics such as fasting mean and standard deviation. These statistics are compared to thresholds to determine the degree of diabetes condition for the patient or the user. These statistics can also be used to adjust medication therapy—for instance, basal insulin or other medications that address fasting glucose levels.

Reconciling meal tags with the meal detection algorithm in accordance with the embodiments of the present disclosure can also be used to refine the default time of day windows to assist users that have different work and rest schedule, such as someone on a night shift. When the pattern changes (e.g. moves from one work shift to another), the report can be updated to adjust accordingly. However, the moving window-based insight on breakfast (as in the first meal since the longest fast of the day) and other meal times can remain properly grouped in spite of the change in what time of day the meals are ingested.

In certain embodiments, data report may be related to the glucose tolerance test. Typically, a glucose tolerance test is administered by measuring the glycemic response to a 75 gram CHO solution administered orally after fasting. This report would rather utilize a number of days of continuous data and determine the statistics that characterize the glycemic response to typical meals for the patient. Statistics may include mean peak glucose deviation and mean time of peak glucose. These statistics are generated based on data segments aligned by the estimated meal start times. These statistics can be compared to thresholds to determine the degree of diabetes condition for the patient. These statistics can also be used to adjust medication therapy—for instance, mean peak glucose deviation may be used to direct changes to meal-time insulin, and mean time of peak glucose could be used to adjust insulin response time settings or to adjust bolus timing.

In certain embodiments, the meal start event and peak meal response event estimation in accordance with the present disclosure provides meal times that can be used to confirm tagged meals and to identify missing tags when analyzing the data to determine a glycemic model from the data.

In certain embodiments, for closed loop control, the estimated meal start events or peak meal response (in real-time) can be used to prompt the user to indicate if they started eating without notifying the closed loop control system of the meal.

In certain embodiments, estimation of meal start events in accordance with the present disclosure is used to prompt the user to ask questions about the meal. One example is prompting the user for mealtime insulin and carbohydrate, if the meal detection suspects a meal has started, but no entry has been logged related to insulin or carbohydrate information. In certain embodiments, the estimated meal start event, after a pre-determined time delay (say 15 minutes), can be used to set up a reminder to dose insulin. In another embodiment, if an estimated meal start event is determined within the most recent hour (or two) of the current acquisition of the sampled glucose data, and if the retrospective analysis of past data warrants checking post-meal glucose (e.g. due to post-meal variability), a reminder can be set to prompt the user to verify the glucose level (for example, using a finger stick test) at a pre-determined duration since the last meal start.

In another embodiment, the user may be provided with a reviewable or selectable option on the user interface of the analyte monitoring device menu structure to try to recall meal starts. The user can scroll through the graph or listing of glucose values, overlaid with potential meal start instances estimated in accordance with the routines described above. In certain embodiments, any confirmed estimate may be stored or identified or marked as a meal event.

In yet another embodiment, the user entered meal markers and estimated start and peak pairs determined in accordance with the present disclosure may be reconciled in conjunction with a healthcare provider, when the data is retrospectively evaluated.

The various methods described herein for performing one or more processes also described herein may be embodied as computer programs (e.g., computer executable instructions and data structures) developed using an object oriented programming language that allows the modeling of complex systems with modular objects to create abstractions that are representative of real world, physical objects and their interrelationships. However, any practicable programming language and/or techniques may be used. The software for performing the inventive processes, which may be stored in a memory or storage device of the computer system described herein, may be developed by a person of ordinary skill in the art based upon the present disclosure and may include one or more computer program products. The computer program products may be stored on a computer readable medium such as a server memory, a computer network, the Internet, and/or a computer storage device. Note that in some cases the methods embodied as software may be described herein with respect to a particular order of operation or execution. However, it will be understood by one of ordinary skill that any practicable order of operation or execution is possible and such variations are contemplated by this specification of the present disclosure.

Various other modifications and alterations in the structure and method of operation of the embodiments of the present disclosure will be apparent to those skilled in the art without departing from the scope and spirit of the present disclosure. Although the present disclosure has been described in connection with certain embodiments, it should be understood that the present disclosure as claimed should not be unduly limited to such embodiments. It is intended that the following claims define the scope of the present disclosure and that structures and methods within the scope of these claims and their equivalents be covered thereby.

Certain embodiments of the present disclosure include performing conditioning on a plurality of data points corresponding to monitored analyte level over a first time period, for each data point, determining a time derivative based on the conditioned plurality of data points, determining optima of acceleration based on the determined time derivatives, removing false carbohydrate intake start and peak carbohydrate intake response pairs having an amplitude below a predetermined level, removing carbohydrate intake start candidate from the most current carbohydrate intake peak response candidate, removing unpaired carbohydrate intake start candidates and signal artifact falsely identified as carbohydrate intake start and carbohydrate intake peak response pair, and refining the identified carbohydrate intake start and peak carbohydrate intake response pairs.

In one aspect, performing conditioning on the plurality of data points corresponding to the monitored analyte level of the first time period includes performing sample data analysis on the plurality of data points to remove questionable data and smoothing the plurality of data points.

One aspect includes outputting an indication associated with a carbohydrate intake start event.

In a further aspect, the carbohydrate intake start event includes a meal start event.

Another aspect includes outputting an indication associated with a peak carbohydrate intake response event.

In a further aspect, the peak carbohydrate intake response event includes a peak meal response event.

Certain embodiments of the present disclosure include a user interface component and one or more processors operatively coupled to the user interface component, the one or more processors configured to perform conditioning on a plurality of data points corresponding to monitored analyte level over a first time period, for each data point, to determine a time derivative based on the conditioned plurality of data points, to determine optima of acceleration based on the determined time derivatives, to remove false carbohydrate intake start and peak carbohydrate intake response pairs having an amplitude below a predetermined level, to remove carbohydrate intake start candidate from the most current carbohydrate intake peak response candidate, to remove unpaired carbohydrate intake start candidates and signal artifact falsely identified as carbohydrate intake start and carbohydrate intake peak response pair, and to remove the identified carbohydrate intake start and peak carbohydrate intake response pairs.

In one aspect, the one or more processors configured to perform conditioning on the plurality of data points corresponding to the monitored analyte level of the first time period, is further configured to perform sample data analysis on the plurality of data points to remove questionable data, and to smooth the plurality of data points.

In another aspect, the one or more processors is configured to output an indication associated with a carbohydrate intake start event on the user interface component.

In one aspect, the carbohydrate intake start event includes a meal start event.

In another aspect, the one or more processors is configured to output an indication associated with a peak carbohydrate intake response event on the user interface component.

In another aspect, the peak carbohydrate intake response event includes a peak meal response event.

1-20. (canceled)

21. A glucose monitoring system for algorithmically removing one or more false carbohydrate intake start candidates and false carbohydrate peak response candidates, the glucose monitoring system comprising:
an on-body device comprising sensor electronics coupled with a glucose sensor, wherein the glucose sensor comprises a portion configured to be positioned through skin of a user and in contact with interstitial fluid, wherein the portion is further configured to sense a glucose level in the interstitial fluid, and wherein the on-body device is configured to transmit data indicative of the glucose level; and
a receiver unit comprising wireless communications circuitry configured to receive the data indicative of the glucose level, the receiver unit further comprising one or more processors coupled with a memory, the memory configured to store instructions that, when executed by the one or more processors, cause the one or more processors to:
determine time derivatives of a plurality of data points corresponding to the data indicative of the glucose level;
determine, based on the time derivatives, a data set comprising a plurality of carbohydrate intake start candidates and a plurality of carbohydrate peak response candidates;
determine whether each carbohydrate intake start candidate of the plurality of carbohydrate intake start candidates has a corresponding carbohydrate peak response candidate of the plurality of carbohydrate peak response candidates;
for each carbohydrate intake start candidate that has a corresponding carbohydrate peak response candidate, pair the each carbohydrate intake start candidate with the corresponding carbohydrate peak response candidate; and
for each carbohydrate intake start candidate that does not have a corresponding carbohydrate peak response candidate, remove the each carbohydrate intake start candidate from the data set.

22. The glucose monitoring system of claim 21, wherein the instructions stored in the memory, when executed by the one or more processors, further cause the one or more processors to:
remove one or more adjacent candidates of a same type.

23. The glucose monitoring system of claim 21, wherein the instructions stored in the memory, when executed by the one or more processors, further cause the one or more processors to:
identify one or more carbohydrate intake start candidates or carbohydrate peak response candidates that comprise a signal artifact, and
remove the one or more carbohydrate intake start candidates or carbohydrate peak response candidates that comprise the signal artifact.

24. The glucose monitoring system of claim 21, wherein the instructions stored in the memory, when executed by the one or more processors, further cause the one or more processors to:
identify one or more carbohydrate intake start candidates having a proximity or a level drop relative to an adjacent carbohydrate peak response candidate that is below a predetermined threshold; and
remove the one or more carbohydrate intake start candidates that has the proximity or the level drop relative to the adjacent carbohydrate peak response candidate that is below the predetermined threshold.

25. The glucose monitoring system of claim 21, wherein the instructions stored in the memory, when executed by the one or more processors, further cause the one or more processors to:
identify one or more carbohydrate intake start candidates or carbohydrate peak response candidates that comprise a difference in amplitude below a predetermined level; and
remove the one or more carbohydrate intake start candidates or carbohydrate peak response candidates that comprise the difference in amplitude below the predetermined level.

26. The glucose monitoring system of claim 21, wherein the instructions stored in the memory, when executed by the one or more processors, further cause the one or more processors to:
refine the each carbohydrate intake start candidate of the plurality of carbohydrate intake start candidates that has the corresponding carbohydrate peak response candidate of the plurality of carbohydrate peak response candidates.

27. The glucose monitoring system of claim 21, wherein the receiver unit further comprises a display, wherein the instructions stored in the memory, when executed by the one or more processors, further cause the one or more processors to:
determine a carbohydrate intake start event based on the each carbohydrate intake start candidate of the plurality of carbohydrate intake start candidates that has the corresponding carbohydrate peak response candidate of the plurality of carbohydrate peak response candidates; and
output an indication associated with the carbohydrate intake start event on the display.

28. The glucose monitoring system of claim 27, wherein the carbohydrate intake start event comprises a meal start event.

29. The glucose monitoring system of claim 21, wherein the receiver unit further comprises a display, wherein the instructions stored in the memory, when executed by the one or more processors, further cause the one or more processors to:
determine a peak carbohydrate response event based on the each carbohydrate intake start candidate of the plurality of carbohydrate intake start candidates that has the corresponding carbohydrate peak response candidate of the plurality of carbohydrate peak response candidates; and
output an indication associated with a peak carbohydrate intake response event on the display.

30. The glucose monitoring system of claim 29, wherein the peak carbohydrate intake response event includes a peak meal response event.

31. The glucose monitoring system of claim 21, wherein the receiver unit further comprises a display, wherein the each carbohydrate intake start candidate of the plurality of carbohydrate intake start candidates that has the corresponding carbohydrate peak response candidate of the plurality of carbohydrate peak response candidates is utilized to confirm an absence or a presence of one or more meal tags manually entered by the user on the receiver unit.

32. The glucose monitoring system of claim 21, wherein the instructions stored in the memory, when executed by the one or more processors, further cause the one or more processors to:
determine a carbohydrate intake start event based on the each carbohydrate intake start candidate of the plurality of carbohydrate intake start candidates that has the corresponding carbohydrate peak response candidate of the plurality of carbohydrate peak response candidates; and
determine a peak carbohydrate response event based on the each carbohydrate intake start candidate of the plurality of carbohydrate intake start candidates that has the corresponding carbohydrate peak response candidate of the plurality of carbohydrate peak response candidates;
wherein the carbohydrate intake start event and the peak carbohydrate response event provide one or more meal times that can be utilized to confirm an absence or a presence of one or more meal tags to determine a glycemic model.

33. The glucose monitoring system of claim 21, wherein the instructions stored in the memory, when executed by the one or more processors, further cause the one or more processors to:
condition the plurality of data points corresponding to the data indicative of the glucose level over a first time period.

34. The glucose monitoring system of claim 33, wherein time derivatives of the plurality of data points are determined based on the conditioned plurality of data points.

35. The glucose monitoring system of claim 21, wherein the instructions stored in the memory, when executed by the one or more processors, further cause the one or more processors to:
remove any outlier data from the plurality of data points corresponding to the data indicative of the glucose level; and
smooth the plurality of data points.

36. The glucose monitoring system of claim 21, wherein determining whether each carbohydrate intake start candidate of the plurality of carbohydrate intake start candidates has a corresponding carbohydrate peak response candidate of the plurality of carbohydrate peak response candidates comprises determining an optima of acceleration.

37. The glucose monitoring system of claim 36, wherein the optima of acceleration is based on the determined time derivatives.

38. The glucose monitoring system of claim 37, wherein determining the optima of acceleration based on the time derivatives includes determining acceleration of the plurality of data points based on the time derivatives.
